(19) United States
(12) Patent Application Publication
(10) Pub. No.: US 2017/0266647 A1
(43) Pub. Date: Sep. 21, 2017

(54) A METHOD OF TREATING A ZEOLITE WITH AN ALUMINUM COMPOUND TO PREPARE A CATALYST FOR THE PRODUCTION OF AROMATICS AND THE CATALYST MADE THEREFROM

(71) Applicant: SABIC Global Technologies B.V., Bergen op Zoom (NL)

(72) Inventors: Sami Muteib Almutairi, Riyadh (SA); Labeeb A. Chaudhary, Riyadh (SA); Majed Mohammed Mussa, Riyadh (SA); Mayank Harishbhai Vyas, Riyadh (SA)

(21) Appl. No.: 15/500,202

(22) PCT Filed: Sep. 2, 2015
(86) PCT No.: PCT/IB2015/056664

§ 371 Date: Jan. 30, 2017

Related U.S. Application Data
(60) Provisional application No. 62/045,229, filed on Sep. 3, 2014.

(51) Int. Cl. B01J 29/40; B01J 29/076; B01J 29/068; B01J 29/072; C07C 2/76; B01J 21/04

(52) U.S. Cl. CPC B01J 29/40; C07C 2/76; B01J 21/04; B01J 29/068; B01J 29/072; B01J 29/076; C07C 2529/40; C07C 2521/08; B01J 2229/18

(57) ABSTRACT

In an embodiment, a process of making a catalyst can comprise contacting a zeolite with an aluminum solution comprising an aluminum compound at a pH of 2 to 6; calcining the zeolite to form the catalyst; wherein the catalyst comprises 0.1 to 5 wt % aluminum based on the total weight of the catalyst excluding any binder or extrusion aide. In an embodiment, a process of aromatizing methane can comprise aromatizing a feed comprising methane in the presence of the catalyst under aromatization conditions.

### TECHNICAL FIELD

The present disclosure relates to a process for preparing a catalyst for the production of aromatics from methane and the catalyst made therefrom.

### BACKGROUND

Aromatic hydrocarbons, for example, benzene, toluene, ethylbenzene, xylenes, and polyaromatic hydrocarbons such as naphthalene, are important commodity chemicals in the petrochemical industry. Currently, aromatics are generally produced from the petroleum-based feedstock by a variety of processes, including catalytic reforming and catalytic cracking. However, as the supply of petroleum feedstock decreases, there is a growing need to find alternative methods for preparing aromatic hydrocarbons.

An alternative method of preparing aromatic hydrocarbons is by the dehydrocyclization of methane as methane is one of the most abundant organic compounds on earth. For example, methane is the major constituent of natural gas; large amounts of methane are trapped in marine sediments as hydrates and in coal shale as coal bed methane; and it can also be derived from a biomass as a biogas.

Accordingly, it would be beneficial to provide a catalyst composition having improved selectivity for converting methane to an aromatic compound, for example, over increased time on stream.

### BRIEF SUMMARY

Disclosed herein are methods for preparing a catalyst for the conversion of methane to aromatics and the catalyst made therefrom.

In an embodiment, a process of making a catalyst comprises contacting a zeolite with an aluminum solution comprising an aluminum compound at a pH of 2 to 6; calcining the zeolite to form a catalyst; wherein the catalyst comprises 0.1 to 5 wt % aluminum based on the total weight of the catalyst excluding any binder or extrusion aide.

In an embodiment, a process of aromatizing methane comprises aromatizing a feed comprising methane in the presence of the catalyst under aromatization conditions.

The above described and other features are exemplified by the following detailed description.

### DETAILED DESCRIPTION

The present disclosure is related to a method of forming a catalyst for the conversion of methane to aromatics. Specifically, the Applicants surprisingly found that a dehydrocyclization catalyst (also referred to herein as the catalyst) prepared by first contacting a support, where the support can be a zeolite, with an aluminum compound at a pH of 2 to 6, for example, followed by contacting with a metal solution comprising a gallium compound, resulted in a catalyst with improved selectivity for aromatics. The final catalyst can comprise 1 to 5 weight percent (wt %) aluminum and can further comprise 0.1 to 3 wt % gallium based on the total weight of the catalyst excluding any binder or extrusion aide. Metal content in the final catalyst can be measured using inductively coupled plasma mass spectrometry (ICP-MS).

The support can comprise an oxide, carbide, and/or nitride of boron, aluminum, silicon, phosphorous, titanium, scandium, chromium, vanadium, magnesium, manganese, iron, zinc, gallium, germanium, yttrium, zirconium, niobium, molybdenum, indium, tin, barium, lanthanum, hafnium, cerium, tantalum, tungsten, a transuranium element, or a combination comprising one or more of the foregoing. The support can comprise a porous material, including but not limited to a microporous crystalline material or a mesoporous material. As used herein, the term “microporous” refers to pores having a diameter of less than 2 nanometers, whereas the term “mesoporous” refers to pores having a diameter form 2 to 50 nanometers. The microporous crystalline support can comprise a silicate, an aluminosilicate, a titanosilicate, an aluminophosphate, a metallophosphate, a silicoaluminophosphate, or a combination comprising one or more of the foregoing.

Specifically, the support can comprise a zeolite that can be any of a number of zeolites, where zeolites are crystalline aluminosilicates with three-dimensional framework containing silica (SiO₄) and alumina (AlO₄) tetrahedra and can be naturally occurring or synthesized. In general, the zeolite framework contains channels and interconnected voids or pores, which can be occupied by cations and water molecules. Depending on the size and geometry of the pores and channels, zeolites can be classified as small, medium, or large pore zeolites, and also as one, two, or three-dimensional pore structure zeolites. The zeolite can comprise ZSM-5, ZSM-22, ZSM-8, ZSM-11, ZSM-12, ZSM-35, or a combination comprising one or more of the foregoing. The zeolite can comprise ZSM-5.

The zeolite can be from a family of pentasil zeolites that contain five membered ring units or pentasil units in the framework structure. Such zeolites include ZSM-5, ZSM-11, ZSM-23, and so on. Silicalite, which contains an isotypic framework of ZSM-5, can also be included. Specifically, the zeolite can be a pentasil zeolite, which contains 10-membered ring pore openings in the structure.

The zeolite can be ZSM-5 also known as MFI (International Zeolite Association nomenclature of ZSM-5). The ZSM-5 zeolite has a two-dimensional pore structure with straight channels (e.g., 5.4 angstroms (Å)×5.6 Å), which are intersected by sinusoidal channels (e.g., 5.1 Å×5.7 Å) with maximum diameter of about 9 Å at the intersection. The ZSM-5 zeolite catalysts and their preparation are described, for example, in U.S. Pat. No. 3,702,886. Such ZSM-5 zeolites are aluminosilicates that contain both silicon and aluminum in the crystalline structure.

The zeolite can have a SiO₂/Al₂O₃ mole ratio (SAR) of, for example, 25 to 1,000, specifically, 200 to 500, and more specifically, 200 to 400. The zeolite can have a SAR of greater than or equal to 40, e.g., 40 to infinity (∝), specifically, 50 to 300. The zeolite can contain up to trace levels of other cations (wherein a trace level is less than or equal to 0.5 wt %, based upon the total weight of the zeolite).

The zeolite can be a germanium zeolite that includes silicon and germanium and optionally aluminum in the crystalline framework of the zeolite structure, for example, the germanium zeolite can be an aluminosilicate zeolite having germanium in the framework and can specifically be a germanium ZSM-5 (Ge-ZSM-5) zeolite. The germanium zeolite can comprise a medium pore zeolite having an average pore size of 5 to 7 Å, a SAR of 40 to infinity (∝), and a germanium content of 0.1 to 10 wt %, specifically, 3.5 to 6.0 wt % based on the total weight of the final catalyst excluding any binder or extrusion aide.

The zeolite can be H⁺ (hydrogen form), having at least a portion of the original cations associated therewith replaced by hydrogen. H⁺ zeolites can be prepared by direct ion exchange with an acid, a base exchange followed by calcinations, by dealumination (such as by acid leaching), or by preparing the zeolite to have an SiO₂/Al₂O₃ ratio of 10 to 100, specifically, 15 to 80, more specifically, 20 to 60. The zeolite can be in NH₄⁺ form having at least a portion of the original cations associated therewith replaced by NH₄⁺. The zeolite can contain no or trace amounts of alkali metal such as sodium (Na) as Na⁺. For example, the final catalyst can contain less than or equal to 0.5 wt % Na₂O, specifically, less than or equal to 0.05 wt % Na₂O, based on the total weight of the final catalyst excluding any binder or extrusion aide.

The zeolite can be prepared using a structure directing agent, which is incorporated in the microporous space of the crystalline network during crystallization, thus controlling the construction of the network and assisting to stabilize the structure through the interactions with, for example, the silicon and aluminum. Structure directing agents (also referred to as structure templating agents), such as tetraethylammonium (TEA⁺), tetrapropylammonium (TPA⁺), or other cations can be present in as-synthesized zeolite. Examples of the structure directing agent are organic amine and quaternary ammonium compounds and salts and cations thereof. The structure directing agent can comprise tetra n-propyl ammonium hydroxide, tetra n-propyl ammonium bromide, tetra n-propyl ammonium chloride, tetraethyl ammonium hydroxide, tetraethylammonium bromide, tetramethylammonium chloride, hexamethyleneimine, 1,4-di(1′4′-diazabicyclo[2.2.2]octane) butane hydroxide, morpholine, cyclohexylamine, diethylethanolamine, N,N′-diisopropyl imidazolium cation, tetrabutylammonium compounds, di-n-propylamine (DPA), tripropylamine, triethylamine (TEA), triethanolamine, piperidine, 2-methylpyridine, N,N-dimethylbenzylamine, N,N-diethylethanolamine, dicyclohexylamine, N,N-dimethyl ethanolamine, choline cation, N,N′-dimethylpiperazine, 1,4-diazabicyclo(2,2,2)octane, 1,6-hexanediamine, N′,N′,N,N-tetramethyl-(1,6)hexanediamine, N-methyldiethanolamine, N-methyl-ethanolamine, N-methyl piperidine, 3-methyl-piperidine, N-methylcyclohexylamine, 3-methylpyridine, 4-methyl-pyridine, quinuclidine, N,N′-dimethyl-1,4-diazabicyclo (2,2,2)octane ion; di-n-butylamine, neopentylamine, di-n-pentylamine, isopropylamine, t-butyl-amine, ethylenediamine, pyrrolidine, 2-imidazolidone, a N-benzyl-1,4-diazabicyclo [2.2.2]octane cation, a 1-[1-(4-chlorophenyl)-cyclopropylmethyl]-1-ethyl-pyrrolidinium cation, a 1-ethyl-1-(1-phenyl-cyclopropylmethyl)-pyrrolidium cation, 1,8-diaminooctane, or a combination comprising one or more of the foregoing. The structure directing agent can comprise tetra-n-propyl ammonium hydroxide (TPAOH).

The structure directing agent can be removed, for example, by heating the zeolite at a temperature of 400 to 600 degrees Celsius (° C.), to result in a zeolite upon which a metal can be added. The heating can be for greater than or equal to 30 minutes (min), specifically, greater than or equal to 2 hours, more specifically, greater than or equal to 3.5 hours.

The zeolite can be contacted with a metal compound in one or more contacting steps, for example, by ion-exchanging (for example, base exchanging), incipient wetness, evaporation, chemical vapor deposition (CVD), impregnation, spray-drying, and physical mixing. The metal compound can be a metal compound, such as an aluminum compound, a gallium compound, a nickel compound, a zinc compound, a molybdenum compound (such as molybdenum oxide, ammonium molybdate, ammonium heptamolybdate, ammonium hexamolybdate, ammonium paramolybdate, and molybdenum oxalate), or a mixture comprising at least one of the foregoing, e.g., to reduce acidity and for methane activation. The compounds can be in the form of a nitrate, a halide (such as a chloride), an oxyhalide (such as an oxychloride), a sulfate, an acetate, an acetylacetonate, a sulfate, an alkoxide, an oxide, an oxalate, a hydroxide, a carbonate, or a combination comprising one or more of the foregoing. It is noted that if the zeolite is contacted with both a zinc compound and a molybdenum compound, then the molybdenum compound can be free of molybdenum oxalate. The zeolite can be ion-exchanged to the extent that most or all of the cations associated with aluminum are the ion-exchanged metal. An example of a monovalent base to aluminum molar ratio in the zeolite after ion exchange is at least 0.9. The ion-exchanged metals are not present in the framework. For example, if the ion-exchanged metal(s) are present in the final catalyst, they can be present within the channels of the zeolite in the final catalyst, and not as part of the framework.

The zeolite can be ion-exchanged by contacting a solution comprising a base metal ion with the zeolite. Each ion-exchange solution can be a 0.01 to 1 molar (M) solution of each of the metal compounds independently and can contact (e.g., be mixed with or can be flowed over or passed through a bed of) the zeolite for a sufficient amount of time to obtain the desired amount of ion-exchange. The ion-exchanging can comprise ion-exchanging to obtain 0.1 to 10.0 wt %, specifically, 0.1 to 5.0 wt %, more specifically, 0.1 to 4.0 wt %, and even more specifically, 5.0 to 3.6 wt % of each of the ion-exchange metals independently in the final catalyst (as determined by ICP-MS), based on the total weight of the final catalyst excluding any binder or extrusion aide. For example, the zeolite can be ion-exchanged with aluminum by making an aqueous slurry of zeolite powder, mixing with a solution comprising a water soluble aluminum compound (such as aluminum nitrate, aluminum chloride, and the like) such that the amount of aluminum in the final catalyst (as determined by ICP-MS) is 0.1 to 5 wt %, for example, 0.1 to 2 wt %, specifically, 0.1 to 0.7 wt %, more specifically, 0.1 to 0.5 wt %, even more specifically, 0.1 to 0.45 wt % based on the total weight of the final catalyst excluding any binder or extrusion aide. Likewise, the zeolite can be ion-exchanged with a gallium compound such that the amount of gallium in the final catalyst (as determined by ICP-MS) is 0.1 to 5 wt %, specifically, 0.1 to 3 wt %, more specifically, 0.5 to 3.0 wt %, even more specifically, 0.5 to 1.5 wt % based on the total weight of the final catalyst excluding any binder or extrusion aide. The final catalyst can comprise 1 to 20 wt %, specifically, 2 to 15 wt %, more specifically, 3 to 10 wt % molybdenum based on the total weight of the final catalyst excluding any binder or extrusion aide.

The process can comprise a single contacting step or multiple contacting steps. For example, the process can comprise first contacting the support, for example, a zeolite, with an aluminum solution comprising an aluminum compound, followed by one or more contacting steps. Following the contacting with an aluminum solution, the zeolite can be contacted with a metal solution comprising a gallium compound, a nickel compound, a zinc compound, a molybdenum compound, or a combination comprising one or more of the foregoing. Likewise, following the contacting with an aluminum solution, the zeolite can be contacted with a gallium compound and/or a nickel compound and/or a zinc compound and/or a molybdenum compound in one or more contacting steps.

The contacting with an aluminum solution can occur at a pH of 2 to 6, specifically, 2.2 to 3.3. The contacting with an aluminum solution can occur at a pH of 3 to 5, specifically, 3.5 to 4.5, more specifically, 3.7 to 4.2. Without being bound by theory, it is believed that depositing the aluminum at such a pH reduces or eliminates damage to the zeolite structure that otherwise occurs as a pH of less than 2. The pH of the aluminum solution can be buffered. The buffer can comprise an ammonium salt, such as ammonium acetate.

Following one or more of the contacting steps, the contacted zeolite can be heated e.g., to a temperature of less than 600° C., specifically, to a temperature of 250 to 350° C.

A noble metal can be deposited as a promoter, which can enhance the performance of the catalyst. The noble metal can be deposited on the heat-treated zeolite, for example, by methods such as ion exchange, impregnation, and incipient wetness impregnation. The noble metal can be added to the heat-treated zeolite as a noble metal compound (e.g., a noble metal salt) that readily dissolves in water. For example, when the metal is platinum, the platinum source can be any applicable platinum source, such as chloroplatinic acid (H₂PtCl₆.6H₂O), tetraamine platinum nitrate ((NH₃)₄Pt(NO₃)₂), or a combination comprising at least one of the foregoing. The noble metal can be present in the final catalyst in an amount of 0.05 to 3 wt % (as measured by ICP-MS), specifically, of 0.15 to 2 wt %, and more specifically, of 0.25 to 1.5 wt %, and even more specifically, of 0.4 to 1.0 wt % based on the total weight of the final catalyst excluding any binder or extrusion aide. The noble metal can be present in the catalyst in an amount of 0.8 to 1.1 wt % (as measured by ICP-MS) based on the total weight of the final catalyst excluding any binder or extrusion aide. In other words, the process can comprise depositing to obtain 0.05 to 3 wt % (as measured by ICP-MS), specifically, of 0.15 to 2 wt %, more specifically, of 0.25 to 1.5 wt %, and even more specifically, of 0.4 to 1.0 wt % platinum in the catalyst, based on the total weight of the final catalyst excluding any binder or extrusion aide. The noble metal can comprise palladium, silver, platinum, gold, iridium, rhodium, ruthenium, or a combination comprising one or more of the foregoing, specifically, the noble metal can comprise platinum.

The final catalyst can comprise a further metal, wherein the further metal can comprise vanadium, chromium, manganese, copper, germanium, niobium, tantalum, tungsten, lead, titanium, silver, lanthanum, neodymium, samarium, iron, cobalt, yttrium, zirconium, hafnium, rhenium, silicon, cerium, strontium, ytterbium, tin, or a combination comprising one or more of the foregoing. The further metal can comprise manganese, tin, boron, lead, copper, iron, chromium, indium, germanium, antimony, bismuth, or a combination comprising one or more of the foregoing. The further metal can comprise germanium. The further metal can comprise germanium, boron, tin, or a combination comprising at least one of the foregoing. The further metal can comprise copper, germanium, niobium, tantalum, silver, lanthanum, samarium, iron, cobalt, hafnium, cerium, or a combination comprising one or more of the foregoing. The further metal can be added in one or more of the above described contacting steps or in one or more of a separate contacting step. The final catalyst can be free of (or can comprise 0 to 0.1 wt %, specifically, 0 to 0.01 wt % of) one or more of rhodium, chromium, vanadium, titanium, manganese, yttrium, zirconium, ruthenium, palladium, lead, neodymium, samarium, tungsten, rhenium, iridium, silicon, strontium, ytterbium, tin, and gold.

After each contacting step independently, the zeolite can be calcined to form the final catalyst. The calcining can be at a temperature of 450 to 750° C. for 0.1 to 100 hours (h), specifically, 2 to 80 hours, more specifically, 10 to 70 hours. The calcining can be done in an inert atmosphere, for example, in nitrogen.

The zeolite, for example, an Al—Ga—Ni—Zn—Mo/ZSM-5 zeolite, can be mixed with a binder that can comprise a solid silica binder and/or a colloidal binder, an extrusion aid, or a combination comprising one or both of the foregoing to form a forming mixture.

The binder can comprise inorganic oxide materials. The binder can comprise an aluminum- or silicon-containing material such as silica, alumina, clay, aluminum phosphate, silica-alumina, quartz, or combinations comprising at least one of the foregoing. The binder can comprise quartz and silica. The binder can comprise a metal oxide (e.g., magnesium oxide, titanium oxide, zirconium oxide, thorium oxide, silicon oxide, and boron oxide); clay (e.g., kaolin and montmorillonite); carbon (e.g., carbon black, graphite, activated carbon, polymers, and charcoal); a metal carbide or nitride (e.g., molybdenum carbide, silicon carbide, and tungsten nitride); a metal oxide hydroxide (e.g., boehmite); or a combination comprising one or more of the foregoing.

The binder can be a silica binder or a substantially silica containing binder, where the substantially silica containing binder means that the binder comprises 0.5 to 15 wt %, specifically, 1 to less than 5 wt %, more specifically, 1 to 4.5 wt % non-silica oxides based on the total weight of the final catalyst including the binder and excluding any extrusion aide.

The binder can comprise at least one colloidal silica binder and at least one solid silica binder. The colloidal silica can be an NH₄⁺ and/or Na⁺ stabilized colloidal silica. Specifically, the colloidal silica can be an ammonium ion stabilized colloidal silica, such as, Ludox™ AS-30, Ludox™ AS-40, Ludox™ SM-30, Ludox™ HS-30, Nalco™ 1034A, available from Nalco Company, or those available from Sigma-Aldrich. The colloidal silica can comprise 30 to 40 wt % silica based on the total weight of the colloidal silica. The colloidal silica can have an average particle size of 1 to 30 nanometer (nm), specifically, 7 to 15 nm. As used herein, particle size is measured along a major axis (i.e., the longest axis) of the particle.

The solid silica can comprise a crystalline silica, an amorphous silica, or a combination thereof. Examples of solid silica include attapulgite, e.g., Min-U-Gel™ commercially available from Active Minerals International, Ultrasil™ commercially available from Degussa Corporation, and Davisil™-643 commercially available from Sigma-Aldrich. The solid silica can have an average particle size of 5 to 30 nm. The solid silica can comprise a high purity solid silica, where ‘high purity solid silica’ is a solid silica that comprises greater than or equal to 70 wt %, specifically, greater than or equal to 80 wt %, more specifically, greater than or equal to 90 wt % of silica oxide, based on the total weight of the solid silica. If a low purity solid silica, such as one that comprises less than 70 wt %, specifically, less than or equal to 66 wt % of silica oxide based on the total weight of the low purity solid silica, is present in the binder, then the colloidal binder should have a particle size of 5 to 20 nm, more specifically, 7 to 15 nm and the forming mixture should be free of an extrusion aide.

The binder can comprise at least one solid binder and a mixture of colloidal binders. For example, the mixture of colloidal binders can include at least 10 wt % of a colloidal binder based on the total weight of the mixture of colloidal binders having an average particle size of 10 to 30 nm, while the remaining colloidal binders can, for example, have an average particle size of 1 to 30 nm. Likewise, the mixture of colloidal binders can comprise at least 20 wt % of a colloidal binder based on the total weight of the mixture of colloidal binders having an average particle size of 10 to 30 nm, while the remaining binders can have an average particle size of 5 to 10 nm. The colloidal binder can have an average surface area of less than or equal to 250 meters squared per gram (m²/g), specifically, 250 to 100 m²/g.

The binder can be present in the final catalyst in an amount of up to 99 wt %, e.g., 1 to 99 wt %, specifically, 10 to 60 wt %, based on the total weight of the final catalyst. The final catalyst can comprise 15 to 50 wt %, specifically, 20 to 40 wt % of silica-containing binder material, based on the total weight of the final catalyst.

The extrusion aide can comprise a partially hydrolyzed polyvinyl alcohol and can be produced commercially by hydrolysis of polyvinyl acetate. When polyvinyl acetate is hydrolyzed, the acetate groups (—COCH₃) are substituted by hydrogen to form alcohol (—OH) groups along the polymer chain. Hereinafter, the term ‘partially hydrolyzed’ refers to a polyvinyl acetate that has been hydrolyzed by less than or equal to 90%. In the partially hydrolyzed polyvinyl alcohol, acetate and alcohol groups are randomly distributed in the polymer chain. The partially hydrolyzed polyvinyl alcohol can have a molecular weight of 500 to 500,000 grams per mole (g/mol), specifically, 10,000 to 200,000 g/mol as measured by gel permeation chromatography such as those commercially available from SIGMA-ALDRICH™. The partially hydrolyzed polyvinyl alcohol can be used in an amount of 0.1 to 5 wt %, specifically, 0.5 to 3 wt %, more specifically, 1 to 2 wt %, based on the total weight of the forming mixture.

The extrusion aide can comprise polyacrylamide. The polyacrylamide can have a molecular weight of 2 to 10 million g/mol, specifically, 2 to 7 million g/mol. The polyacrylamide can be used in an amount of 0.1 to 5 wt %, specifically, 0.5 to 3 wt %, more specifically, 1 to 2 wt %, based on the total weight of the forming mixture. An example of a commercially available source of polyacrylamide is that sold under the trademark CYFLOC™ N-300 LMW Flocculent available from Cytec, West Paterson, N.J., which is a polyacrylamide having a molecular weight of 2 to 5 million g/mol.

The forming mixture can be formed into a shaped body (also referred to as a formed zeolite) by various forming processes such as pelletizing, tableting, extruding, and any other technique of forming the forming mixture into a shape, as well as a combination comprising at least one of the foregoing processes. The resulting shaped body can be, for example, pellets and/or tablets. The shaped body can have cross-sections that are, for example, circular, oval, oblong, square, rectangular, diamond, polygonal, or a combination comprising one or more of the foregoing. Specific examples include cylindrically shaped extrudates, such as 1/16 inch (1.6 millimeter (mm)) or ⅛ inch (3.2 mm) cylindrically shaped extrudates. The shaped body can be a sphere with an average diameter measured on a major axis of 5 micrometers to 15 mm. The shaped body can be an extrudate with an average diameter measured on a major axis of 0.5 to 10 mm and an average length of 1 to 15 mm. The forming can be performed at temperatures of less than or equal to 350° C.

After the forming mixture is formed into a shaped body, the shaped body can be calcined in an oxygen containing environment at a temperature not exceeding 350° C. and/or activated at a temperature less than 600° C. in a reducing environment (e.g., under flow of H₂) to result in the final catalyst. The shaped body can be heated in an oxygen containing environment to a temperature of 100 to 350° C. for greater than or equal to 0.5 h, specifically, greater than or equal to 1 h, more specifically, greater than or equal to 2 h. The shaped body can be heated in an oxygen containing environment for 0.5 to 20 h.

The catalyst can be used to convert alkanes to aromatics (such as benzene, toluene, ethylbenzene, xylenes, polyaromatic hydrocarbons such as naphthalene, or a combination comprising one or more of the foregoing) in a dehydrocyclization (also referred to as a dehydroaromatization) step by introducing a feed comprising the alkane to the catalyst under aromatization conditions. The feed can comprise C₁₋₁₂ hydrocarbons, specifically, C₁₋₅ hydrocarbons, more specifically, C₁₋₂ hydrocarbons, either alone or as components in a mixture. The feed can be a methane feed, for example, that has a methane feed source of natural gas, a coal bed, a landfill, agricultural or municipal waste fermentation cite, or a refinery gas source. The feed can comprise 80 to 99.9 mole percent (mol %), specifically, 90 to 99.9 mol %, more specifically, 97 to 99 mol % methane based on the total moles of feed.

In addition to methane, the feed can comprise carbon dioxide, carbon monoxide, hydrogen, steam, an inert gas (such as argon), or a combination comprising one or more of the foregoing. The feed can comprise 0.1 to 10 mol %, specifically, 1 to 3 mol % carbon dioxide based on the total amount of feed. The feed can comprise 0 to 10 mol % of an inert gas based on the total amount of feed. The feed can comprise 0.1 to 20 mol %, specifically, 1 to 6 mol % carbon monoxide based on the total amount of feed. The feed can comprise 0.1 to 10 mol %, specifically, 1 to 5 mol % steam based on the total amount of feed. The feed can comprise 0.1 to 20 mol %, specifically, 1 to 5 mol % hydrogen based on the total amount of feed. The feed can comprise aromatics, for example, from a recycle stream. Any nitrogen and/or sulfur impurities present in the feed can be removed or reduced to low levels prior to the converting. For example, the feed can contain less than 100 parts per million by weight (ppm), specifically, less than 10 ppm, more specifically, less than 1 ppm each of nitrogen and sulfur compounds independently. In other words, the feed can be free of nitrogen and/or sulfur compounds (i.e., the compounds are not measurable using current measurement standards).

The converting can be done in the absence of molecular oxygen, O₂. For example, in the presence of less than 100 ppm, specifically, less than 10 ppm, more specifically, less than 1 ppm of molecular oxygen. In other words, the feed can be free of molecular oxygen (i.e., the molecular oxygen is not measurable using current measurement standards).

The converting can occur at a temperature of 400 to 1,200° C., specifically, 600 to 950° C., more specifically, 700 to 760° C. The converting can occur at a pressure of 1 to 1,000 kiloPascal (kPa), specifically, 10 to 500 kPa, more specifically, 50 to 200 kPa. The contact between the feed and the catalyst can be at a gas hourly space velocity (GHSV) of 0.01 to 1,000 hour⁻¹ (1/h), specifically, 0.1 to 500 1/h, more specifically, 1 to 20 1/h.

The converting can occur in one or more of a fixed bed, a moving bed, or a fluidized bed. The feedstock can be contacted in one or more reaction zones with a moving bed of catalyst, wherein the feedstock flows countercurrent to the direction of movement of the catalyst. The reaction zone can comprise a settling bed reactor that can be a vertically disposed reactor, in which the catalyst enters at or near the top of the reactor and flows under gravity to form a catalyst bed, while the feed enters the reactor at or near the base of the reactor and flows upward through the catalyst bed. Alternatively, the reaction zone can comprise a plurality of series-connected fluidized bed reactors, in which particulate catalyst is cascaded in one direction from one reactor to the next adjacent reactor in the series, while the feed is passed through and between the reactors in the opposite direction.

### EXAMPLES

### Example 1

### Catalyst Preparation

The catalyst was prepared by adding 10 grams (g) of ZSM-5 zeolite to a beaker. An aluminum nitrate solution was prepared in a separate beaker and the pH was adjusted using an ammonium acetate buffer solution to a value of 4. The aluminum nitrate solution was added to the ZSM-5 zeolite under stirring at 60° C. for 30 minutes. The material is dried in air oven at 100° C. overnight to form an Al/ZSM-5 zeolite. 0.6 g of the Al/SZM-5 zeolite was mixed with 1.8 g of quartz and 0.6 g of silica and heated to 700° C. under nitrogen for 40 minutes to form Catalyst 1.

### Example 2

### Catalyst Performance

The catalyst of Example 1 was then tested for aromatics selectivity under dehydrocyclization conditions at a temperature of 700° C., gas hour space velocity (GHSV) of 750 milliliters per hour per gram of catalyst, and a pressure of 1 atm. The conversion reaction was performed in a continuous flow quartz tubular fixed-bed reactor, having an inner diameter of 10 mm. Specifically, a feed comprising 95 mol % methane and 5 mol % argon was introduced to 3 g of Catalyst 1 in the reactor for 90 minutes. The conversion, yield, and selectivity data is tabulated in Table 1, where TOS stands for Time on Stream.

TABLE 1

| | Benzene | Benzene | Benzene | Aromatics |
|---|---|---|---|---|
| TOS | Conversion | Selectivity | Yield | Selectivity |
| (h) | (mol %) | (mol %) | (mol %) | (mol %) |
| 2.1 | 11.1 | 94 | 10.4 | 97 |
| 3.6 | 11.6 | 94 | 10.9 | 98 |
| 4.0 | 12.4 | 75 | 9.3 | 98 |
| 20.2 | 11.9 | 75 | 8.9 | 96 |
| 21.7 | 11.0 | 76 | 8.3 | 96 |
| 23.8 | 10.6 | 74 | 7.8 | 96 |
| 24.8 | 10.4 | 73 | 7.6 | 95 |
| 44.5 | 6.6 | 70 | 4.6 | 90 |
| 45.7 | 6.0 | 72 | 4.3 | 88 |
| 46.8 | 6.0 | 70 | 4.2 | 88 |
| 52.2 | 5.6 | 65 | 3.6 | 87 |
| 67.9 | 3.9 | 62 | 2.4 | 81 |
| 69.1 | 3.4 | 65 | 2.2 | 81 |

Table 1 shows that the catalyst has a high aromatics selectivity of 81 mol % and a high benzene selectivity of 65 mol % after almost 70 hours on stream.

Set forth below are some embodiments of the present process.

Embodiment 1: a process of making a catalyst comprising: contacting a zeolite with an aluminum solution comprising an aluminum compound at a pH of 2 to 6; calcining the zeolite to form a catalyst; wherein the catalyst comprises 0.1 to 5 wt % aluminum based on the total weight of the catalyst excluding any binder or extrusion aide.

Embodiment 2: the process of Embodiment 1, wherein the contacting further comprises contacting the zeolite with a gallium compound.

Embodiment 3: the process of any of Embodiments 1-2, wherein the contacting with the gallium compound further comprises contacting the zeolite with the gallium compound after the contacting with the aluminum solution; and wherein the catalyst comprises 0.1 to 3 wt % gallium based on the total weight of the catalyst excluding any binder or extrusion aide.

Embodiment 4: the process of any of Embodiments 1-3, wherein the aluminum compound comprises aluminum nitrate.

Embodiment 5: the process of any of Embodiments 1-4, wherein the aluminum solution further comprises a buffer.

Embodiment 6: the process of Embodiment 5, wherein the buffer comprises ammonium acetate.

Embodiment 7: the process of any of Embodiments 1-6, wherein the pH is 2.2 to 3.2.

Embodiment 8: the process of any of Embodiments 1-7, wherein the pH is 3 to 5, specifically, 3.5 to 4.5, more specifically, 3.7 to 4.2.

Embodiment 9: the process of any of Embodiments 1-8, wherein the catalyst comprises 0.1 to 3 wt % gallium.

Embodiment 10: the process of any of Embodiments 1-9, wherein the catalyst comprises 0.5 to 1.5 wt % gallium.

Embodiment 11: the process of any of Embodiments 1-10, further comprising contacting with one or more of a nickel compound, a zinc compound, and a molybdenum compound, in one or more contacting steps.

Embodiment 12: the process of Embodiment 11, comprising contacting the nickel compound, the zinc compound, and the molybdenum compound, in separate contacting steps.

Embodiment 13: the process of Embodiment 11, comprising contacting the nickel compound, the zinc compound, and the molybdenum compound, in a single contacting step.

Embodiment 14: the process of any of Embodiments 1-13, further comprising contacting with a further metal, wherein the further metal comprises vanadium, chromium, manganese, copper, germanium, niobium, tantalum, tungsten, lead, titanium, silver, lanthanum, neodymium, samarium, iron, cobalt, yttrium, zirconium, hafnium, rhenium, silicon, cerium, strontium, ytterbium, tin, or a combination comprising one or more of the foregoing. Specifically, the further metal comprises manganese, tin, boron, lead, copper, iron, chromium, indium, germanium, antimony, bismuth, or a combination comprising one or more of the foregoing

Embodiment 15: the process of Embodiment 14, wherein the further metal comprises germanium, boron, tin, or a combination comprising at least one of the foregoing or copper, germanium, niobium, tantalum, silver, lanthanum, samarium, iron, cobalt, hafnium, cerium, or a combination comprising one or more of the foregoing.

Embodiment 16: the process of any of Embodiments 1-15, further comprising contacting the zeolite with a noble metal, wherein the noble metal comprises palladium, silver, platinum, gold, iridium, rhodium, ruthenium, or a combination comprising one or more of the foregoing. Specifically, the noble metal comprises platinum.

Embodiment 17: the process of Embodiment 16, wherein the noble metal is present in the catalyst in an amount of 0.05 to 3 wt %, or 0.15 to 2 wt %, or 0.25 to 1.5 wt %, or 0.4 to 1.0 wt % based on the total weight of the final catalyst excluding any binder or extrusion aide

Embodiment 18: the process of any of Embodiments 1-17, further comprising mixing the catalyst with a binder and to produce a bound catalyst.

Embodiment 19: the process of Embodiment 18, wherein the binder comprises one or both of a solid silica binder and a colloidal binder.

Embodiment 20: the process of any of Embodiments 1-19, wherein the catalyst comprises 0.1 to 2 wt % aluminum.

Embodiment 21: the process of any of Embodiments 1-20, wherein the catalyst comprises 0.1 to 0.7 wt % aluminum.

Embodiment 22: the process of any of Embodiments 1-21, further comprising contacting the zeolite with a gallium compound, wherein the gallium compound is in a metal solution, and wherein the metal solution further comprises a nickel compound, a zinc compound, a molybdenum compound, or a combination comprising one or more of the foregoing.

Embodiment 23: the process of any of Embodiments 1-22, wherein the zeolite comprises ZSM-5, ZSM-22, ZSM-8, ZSM-11, ZSM-12, ZSM-35, or a combination comprising one or more of the foregoing.

Embodiment 24: the process of any of Embodiments 1-23, wherein the zeolite comprises ZSM-5.

Embodiment 25: the process of any of Embodiments 1-24, wherein the zeolite has a SAR of 25 to 1,000, specifically, 200 to 500, and more specifically, 200 to 400 or wherein the zeolite has a SAR of greater than or equal to 40, e.g., 40 to infinity (∝), specifically, 50 to 300.

Embodiment 26: the process of any of Embodiments 1-25, wherein the zeolite has a framework, wherein the framework comprises germanium.

Embodiment 27: the process of any of Embodiments 1-26, wherein the zeolite is a hydrogen form zeolite or a NH₄⁺ form zeolite.

Embodiment 28: the process of any of Embodiments 1-27, wherein the zeolite contains no or trace amounts of alkali metal such as sodium.

Embodiment 29: the process of any of Embodiments 1-28, wherein the contacting comprises ion-exchanging.

Embodiment 30: the process of Embodiment 29, wherein a monovalent base to aluminum molar ratio in the zeolite after ion exchanging is at least 0.9.

Embodiment 31: the process of any of Embodiments 1-30, wherein the catalyst comprises 1 to 20 wt %, specifically, 2 to 15 wt %, more specifically, 3 to 10 wt % molybdenum based on the total weight of the final catalyst excluding any binder or extrusion aide

Embodiment 32: a catalyst made by any of Embodiments 1-31.

Embodiment 33: a process of aromatizing methane comprising: aromatizing a feed comprising methane in the presence of the catalyst of Embodiment 32 under aromatization conditions.

Embodiment 34: the process of Embodiment 33, wherein the feed further comprises ethane, carbon dioxide, carbon monoxide, hydrogen, steam, or a combination comprising one or more of the foregoing.

Embodiment 35: the process of any of Embodiments 33-34, wherein the feed comprises 80 to 99.9 mol % methane.

Embodiment 36: the process of any of Embodiments 33-35, wherein the feed comprises one or more of 0.1 to 10 mol % carbon dioxide, 0.1 to 20 mol % carbon monoxide, 0.1 to 10 mol % steam and 0.1 to 20 mol % hydrogen.

Embodiment 37: the process of any of Embodiments 33-36, wherein the feed comprises an inert gas.

Embodiment 38: the process of any of Embodiments 33-37, wherein the feed comprises less than 100 ppm, specifically, less than 10 ppm, more specifically, less than 1 ppm each of nitrogen and sulfur compounds independently.

Embodiment 39: the process of any of Embodiments 33-38, wherein the aromatizing is performed in the presence of less than 100 ppm, specifically, less than 10 ppm, more specifically, less than 1 ppm of molecular oxygen.

Embodiment 40: the process of any of Embodiments 33-39, wherein the aromatizing forms benzene, toluene, ethylbenzene, xylenes, polyaromatic hydrocarbons such as naphthalene, or a combination comprising one or more of the foregoing.

Embodiment 41: the process of any of Embodiments 33-40, wherein the aromatizing occurs at one or more of a temperature of 400 to 1,200° C., specifically, 600 to 950° C., more specifically, 700 to 760° C.; at a pressure of 1 to 1,000 kPa, specifically, 10 to 500 kPa, more specifically, 50 to 200 kPa; and a gas hourly space velocity GHSV of 0.01 to 1,000 hour⁻¹, specifically, 0.1 to 500 1/h, more specifically, 1 to 20 1/h.

Embodiment 42: the process of any of Embodiments 33-41, wherein the aromatizing occurs in a fixed bed, a moving bed, or a fluidized bed.

Embodiment 43: use of the catalyst of Embodiment 32 in aromatizing methane.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants, or species used in the prior art compositions or that are otherwise not necessary to the achievement of the function and/or objectives of the present invention.

All ranges disclosed herein are inclusive of the endpoints, and the endpoints are independently combinable with each other (e.g., ranges of “up to 25 wt %, or, more specifically, 5 to 20 wt %”, is inclusive of the endpoints and all intermediate values of the ranges of “5 to 25 wt %,” etc.). “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment,” “another embodiment,” “an embodiment,” and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While particular embodiments have been described, alternatives, modifications, variations, improvements, and substantial equivalents that are or may be presently unforeseen may arise to Applicants or others skilled in the art. Accordingly, the appended claims as filed and as they may be amended are intended to embrace all such alternatives, modifications variations, improvements, and substantial equivalents.

Disclosure of a narrower range in addition to a broader range is not a disclaimer of the broader range.

All references disclosed herein are incorporated in their entirety by reference.

This application claims priority to U.S. Application No. 62/045,299, which was filed on 3 Sep. 2014, the contents of which are incorporated in their entirety by reference.

I/We claim:

1. A process of making a catalyst comprising:
contacting a zeolite with an aluminum solution comprising an aluminum compound at a pH of 2 to 6;
calcining the zeolite after the contacting to form the catalyst;
wherein the catalyst comprises 0.1 to 5 wt % aluminum based on the total weight of the catalyst excluding any binder or extrusion aide.

2. The process of claim 1, wherein the contacting further comprises contacting the zeolite with a gallium compound.

3. The process of claim 2, wherein the contacting with the gallium compound further comprises contacting the zeolite with the gallium compound after the contacting with the aluminum solution; and wherein the catalyst comprises 0.1 to 3 wt % gallium based on the total weight of the catalyst excluding any binder or extrusion aide.

4. The process of claim 1, wherein the aluminum compound comprises aluminum nitrate.

5. The process of claim 1, wherein the aluminum solution further comprises a buffer.

6. The process of claim 1, wherein the pH is 2 to 4, or 2.2 to 3.2.

7. The process of claim 1, wherein the catalyst comprises 0.5 to 3 wt %, or 0.5 to 1.5 wt % gallium.

8. The process of claim 1, further comprising contacting the zeolite with one or more of a nickel compound, a zinc compound, and a molybdenum compound, in one or more contacting steps.

9. The process of claim 1, further comprising contacting the zeolite with a further metal, wherein the further metal comprises vanadium, chromium, manganese, copper, germanium, niobium, tantalum, tungsten, lead, titanium, silver, lanthanum, neodymium, samarium, iron, cobalt, yttrium, zirconium, hafnium, rhenium, silicon, cerium, strontium, ytterbium, tin, or a combination comprising one or more of the foregoing.

10. The process of claim 1, further comprising contacting the zeolite with a noble metal, wherein the noble metal comprises palladium, silver, platinum, gold, iridium, rhodium, ruthenium, or a combination comprising one or more of the foregoing.

11. The process of claim 1, further comprising mixing the catalyst with a binder and to produce a bound catalyst.

12. The process of claim 1, wherein the catalyst comprises 0.1 to 2 wt % aluminum.

13. The process of claim 1, wherein the catalyst comprises 0.1 to 0.7 wt % aluminum.

14. The process of claim 1, further comprising contacting the zeolite with a gallium compound, wherein the gallium compound is in a metal solution, and wherein the metal solution further comprises a nickel compound, a zinc compound, a molybdenum compound, or a combination comprising one or more of the foregoing.

15. A catalyst made by claim 1.

16. A process of aromatizing methane comprising: aromatizing a feed comprising methane in the presence of the catalyst of claim 15 under aromatization conditions.

17. The process of claim 16, wherein the feed further comprises ethane, carbon dioxide, carbon monoxide, hydrogen, steam, or a combination comprising one or more of the foregoing.

18. The process of claim 16, wherein the feed comprises 80 to 99.9 mol % methane.

19. The process of claim 16, wherein the feed comprises one or more of 0.1 to 10 mol % carbon dioxide, 0.1 to 20 mol % carbon monoxide, 0.1 to 10 mol % steam and 0.1 to 20 mol % hydrogen.

20. (canceled)

21. The process of claim 1, wherein the zeolite comprises ZSM-5, ZSM-22, ZSM-8, ZSM-11, ZSM-12, ZSM-35, or a combination comprising one or more of the foregoing.
